(19) United States
(12) Patent Application Publication
(10) Pub. No.: US 2025/0241518 A1
(43) Pub. Date: Jul. 31, 2025

(54) ENDOSCOPIC INSTRUMENT

(71) Applicant: Richard Wolf GmbH, Knittlingen (DE)

(72) Inventor: Sebastian FREY, Waghäusel (DE)

(21) Appl. No.: 18/856,183

(22) PCT Filed: Apr. 12, 2023
(86) PCT No.: PCT/DE2023/200075

§ 371 Date: Oct. 11, 2024

(30) Foreign Application Priority Data
Apr. 14, 2022 (DE) 10 2022 203 780.4

(51) Int. Cl. A61B 1/00; A61B 1/018

(52) U.S. Cl. CPC A61B 1/00135; A61B 1/00066; A61B 1/00128; A61B 1/018

(57) ABSTRACT

An endoscopic instrument includes a handle and an endoscope shank (8) which coming from the handle (6) extends distally. The endoscope shank is provided with a spacer element (16), which coming from the handle (6), extends distally in the circumferential region of the endoscope shank (8) and is linearly movably mounted in the handle (6). An endoscopic system includes such an instrument.

### CROSS REFERENCE TO RELATED APPLICATIONS

This application is a United States National Phase Application of International Application PCT/DE2023/200075, filed Apr. 12, 2023, and claims the benefit of priority under 35 U.S.C. § 119 of German Application DE 10 2022 203 780.4, filed Apr. 14, 2022, the entire contents of which are incorporated herein by reference.

### TECHNICAL FIELD

The present invention pertains to an endoscopic instrument as well as to an endoscopic system with such an endoscopic instrument.

### BACKGROUND

An endoscopic system which comprises an endoscope and a working sleeve is known from DE 10 2012 220 651 B4. An endoscope intermediate piece which can be axially adjusted in its length is arranged between the endoscope and the working sleeve. By way of this, the endoscope can be axially positioned in the working sleeve. With regard to the intermediate piece, it is the case of a separate, loose component, which complicates the assembly and the handling.

### SUMMARY

It is an object of the invention to provide an endoscopic instrument which is simpler to handle and which permits an axial positioning of the instrument in a working shank.

This object is achieved by an endoscopic instrument with the features which are specified in the description as well as by an endoscopic system with the features.

The endoscopic instrument comprises a handle and an endoscope shank which coming from the handle extends distally. The handle is designed for gripping, holding and handling the endoscope. The handle in the usual manner can comprise illumination connections and/or rinsing/suction connections and openings to working channels and in particular a viewing opening into optics or connections for a camera system or the like. In a known manner, for example at least one working channel, rinsing channels, illumination devices and picture transmission systems such as for example an optics shank are arranged in the endoscope shank. According to the invention, a spacer element is provided, said spacer element coming from the handle extending distally in the circumferential region of the endoscope shank. I.e. the spacer element is arranged in the circumferential region of the endoscope shank. Particularly preferably, the spacer element can be arranged at several circumferential sides of the endoscope shank or surround the endoscope shank over the complete circumference. According to the invention, the spacer element is mounted in the handle in a linearly movable manner. Herein, the mounting is preferably located in the inside of the handle, i.e. in a region of the handle which to the outside is surrounded by a structure or wall. The spacer element is preferably connected to the handle in a manner which does not require and preferably does not render possible a separation from the handle and spacer element in normal use. This simplifies the handling of the instrument since there are less individual parts. The spacer element serves for axially positioning the endoscopic instrument in the working shank on inserting the endoscopic instrument into a working shank. For this, the spacer element can preferably come to bear on a contact surface at the proximal end of the working shank.

Preferably, the spacer element is linearly movably mounted in the handle in a manner such that a distance of the spacer element to the distal end of the endoscope shank can be changed. This means that the spacer element can be moved forwards and backwards in the axial direction in the mounting in the handle, so that the distal end of the spacer element projects differently far from the handle, by which means the distance to the distal end of the endoscope shank also changes. If the spacer element bears on a working shank, then the distal end of the endoscope shank is positioned relative to the distal end of the working shank.

The spacer element preferably comprises a contact surface which is designed to come to bear on a proximal axial end of a working shank. The contact surface is further preferably formed by the distal end face of the spacer element and preferably extends normally, i.e. at right angles to the longitudinal axis of the endoscope shank. The spacer element can preferably come to bear with this contact surface on the proximal end of the working shank, at which a corresponding counter contact-surface is present. The counter-contact surface for example is herein formed by the proximal face edge or end face of the working shank.

Particularly preferably, the spacer element is designed as a spacer sleeve which circumferentially surrounds the endoscope shank. The spacer sleeve expediently has a circular cross section and the middle axis of the spacer sleeve extends parallel to the longitudinal axis of the endoscope shank or along the longitudinal axis of the endoscope shank. The spacer element or the spacer sleeve is herein linearly movable along its longitudinal axis, i.e. the mounting in the handle is designed such that it permits a linear movement of the spacer sleeve along its longitudinal axis. Given the design as a spacer sleeve, the previously described contact surface is preferably an annular contact surface which is formed by the distal end face of the spacer sleeve and preferably extends normally to the longitudinal axis of the spacer element.

According to a further possible embodiment of the invention, the spacer element comprises a thread which is in engagement with a counter-thread in the handle in a manner such that the spacer element is linearly movable by way of rotation via the threaded engagement between the thread and counter-thread. Herein, the thread is arranged such that its middle axis or rotation axis extends parallel to the longitudinal axis of the endoscope shank or along the longitudinal axis of the endoscope shank, so that the spacer element can be moved forwards and backwards parallel to the endoscope shank by way of rotation in the thread. Given the design of the spacer element as a spacer sleeve, the rotation axis of the thread is preferably identical to the middle or longitudinal axis of the spacer sleeve, so that the spacer sleeve as a whole can be rotated in the thread and herein displaces in the linear direction in accordance with the pitch of the thread.

The thread is particularly preferably designed as an inner thread in the inside of the spacer sleeve. The thread is therefore protected in the inside by the spacer sleeve. The thread can be designed as a multi-flight thread, in order to achieve a large as possible travel with little revolution.

The counter-thread is preferably designed as an outer thread preferably on the outer circumference of the endoscope shank or on a distal section of the handle which connects onto the proximal end of the endoscope shank. The longitudinal axis of the counter-thread herein extends parallel to the longitudinal axis of the endoscope shank or along the longitudinal axis of the endoscope shank. This means that the counter-thread does not necessarily need to be located concentrically to the longitudinal axis of the endoscope shank, but can be offset by a certain amount in the radial direction. In particular, this lends itself if the endoscope shank is not centrally led in the working sleeve and/or if the endoscope shank has no circular cross section. The counter-thread can be arranged in the handle such that it is covered by the spacer sleeve and/or parts of the outer wall of the handle, so that it is protected from damage and/or contamination. Furthermore, a risk of injury to the user, for example by way of getting pinched, is prevented.

According to a further preferred embodiment, the spacer sleeve comprises a grip region on its outer periphery, preferably in the region of its distal end. Such a grip region can be designed for example as a corrugation, in the form of projections or the like. Herein, the grip region preferably comprises sections which are designed as flattenings and/or projections and do not extend precisely in the circumferential direction with respect to the rotation axis of the spacer sleeve. This permits the grip region to be able to be gripped and for a torque to be able to be transmitted via this onto the spacer sleeve for its rotation in the thread. The arrangement of the grip region at the distal end has the advantage that the spacer sleeve can be well gripped in all possible axial positions.

According to a further preferred embodiment of the invention, the handle comprises a wall or wall structure which surrounds a receiving space which is open to the distal end of the handle and into which the spacer element engages or in which the spacer element is mounted. In particular, the wall structure can thus reliably surround the part of the spacer element which is received in the handle and in the inside of the handle surround or cover the mounting region of the spacer element, for example an outer thread.

The spacer element can extend out of the handle through the opening of the receiving space which is directed towards the distal end of the handle, and in particular be moved out of the handle to a differently far extent, depending on its axial positioning.

The spacer element is herewith preferably linearly movable out of the receiving space and into the receiving space, in a manner such that its projects differently far in the distal direction, in particular with respect to the distal end of the handle. In a completely retracted position, in which the spacer element is situated in its position which is situated furthest proximally, the spacer element for the larger part is arranged in the inside of the receiving space which is surrounded by the wall structure, which is to say in the inside of the handle. In the axial position of the spacer element which is situated most distally, the spacer element projects further with respect to the distal end of the wall structure, i.e. it is moved maximally out of the receiving space.

The wall structure preferably comprises openings. Herein, this can preferably be a multitude of openings or through-openings which further preferably are arranged in a manner distributed essentially uniformly over the wall surface or surface of the wall structure. Hence a grid structure with a multitude of openings is particularly preferably created. The openings form a perforation which permits a good rinsing also of the receiving space and in particular of a thread in which the spacer element is mounted. The wall structure can simultaneously have a shaping which permits the hand to be able to optimally embrace this region of the handle too.

Apart form the previously described endoscopic instrument, the invention encompasses an endoscopic system with such an endoscopic instrument as has been described previously. Apart from the endoscopic instrument, the endoscopic system also comprises a working shank into which the endoscopic instrument can be inserted from the distal end in a manner such that the spacer element comes to bear on the proximal end of the working shank. The endoscope shank can be positioned in the inside of the working shank to a different extent in the axial direction by way of the axial displacement of the spacer element. The further the spacer element, for example a spacer sleeve is moved into the inside of the handle, the closer is the complete handle situated towards the proximal end of the working shank, by which means the endoscope shank and its distal end is displaced further in the distal direction with respect to the working shank. Herein, the distal end of the endoscope shank can be moved out of the distal end of the working shank to a different extent, depending on how far the spacer element is moved out of the handle.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a lateral view of an endoscope system according to the invention, in a first positioning of the spacer element;

FIG. 2 is a lateral view of the endoscope system, in a second axial positioning of the spacer element;

FIG. 3 is a lateral view of the endoscopic instrument in the endoscope system;

FIG. 4 is a detailed view of the distal end of the handle of the endoscopic instrument in the first axial positioning; and

FIG. 5 is a detailed view of the distal end of the handle in the second axial positioning of the spacer element.

### DESCRIPTION OF PREFERRED EMBODIMENTS

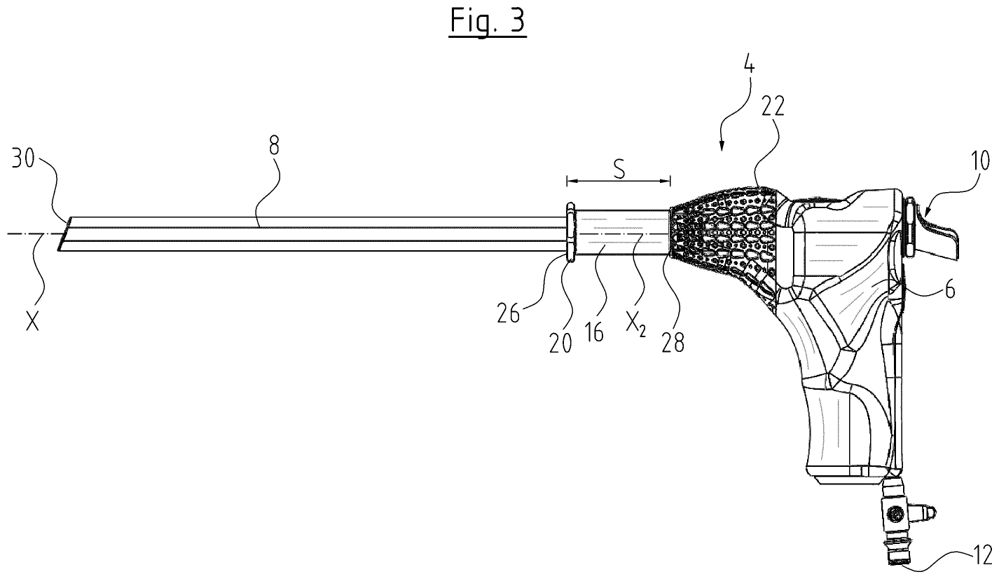

Referring to the drawings, the endoscopic system which is shown here as an example comprises a working shank 2 as well as an endoscopic instrument 4 which is arranged therein, as is shown in FIG. 3. The endoscopic instrument 4 comprises a handle 6 which is arranged at the proximal end, as well as an endoscope shank 8 which extends away from the handle 6 in the distal direction. The endoscope shank in its inside in the usual manner comprises a working channel, one or more rinsing channels, a picture transmission system and/or for example fiber optics for illumination or light diodes for illumination. The working channel is open towards the proximal end of the handle 6. An insertion opening 10 is formed there, through which insertion opening instruments can be inserted into the inside of the endoscope shank 8. Moreover, at least one rinsing/suction connection 12 is arranged on the handle and is in connection with a rinsing channel in the inside of the endoscope shank 8. In this embodiment example, the working shank 2 also comprises a rinsing/suction connection 14 which is in connection with the interior of the working shank 2 so that the free space between the inner circumference of the working shank 2 and the outer circumference of the endoscope shank 8 can likewise serve as a rinsing channel.

According to the invention, the handle 6 comprises an integrated spacer element in the form of a spacer sleeve 16 which can be moved out of the handle 6 in the distal direction to a different extent. The spacer sleeve 16 comprises a thread 17 on its inner circumference, said thread extending into a counter thread 18 on the outer circumference of a section of the handle 6 which connects proximally onto the endoscope shank 8. The spacer sleeve 16 at its distal end comprises a grip region 20, at which the spacer sleeve 16 can be gripped and be rotated on the counter-thread 18. Herein, it simultaneously moves forwards or backwards in the axial direction in the direction of its rotation axis and longitudinal axis X₂, depending on the rotation direction. In this embodiment example, the rotation and longitudinal axis X₂ of the spacer sleeve 16 is offset parallel to the longitudinal axis X of the endoscope shank 8.

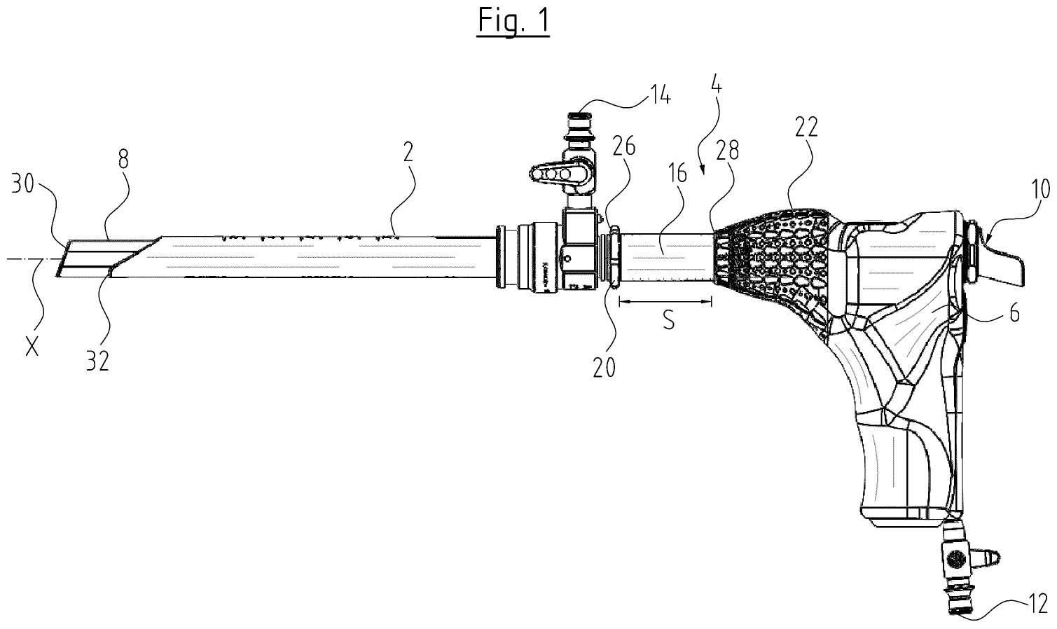

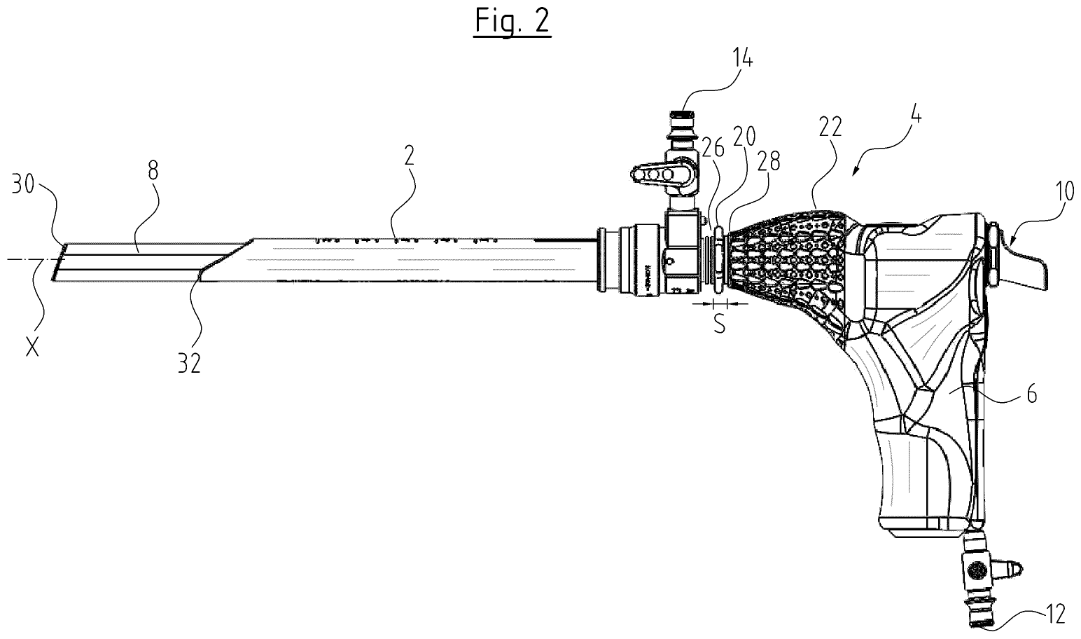

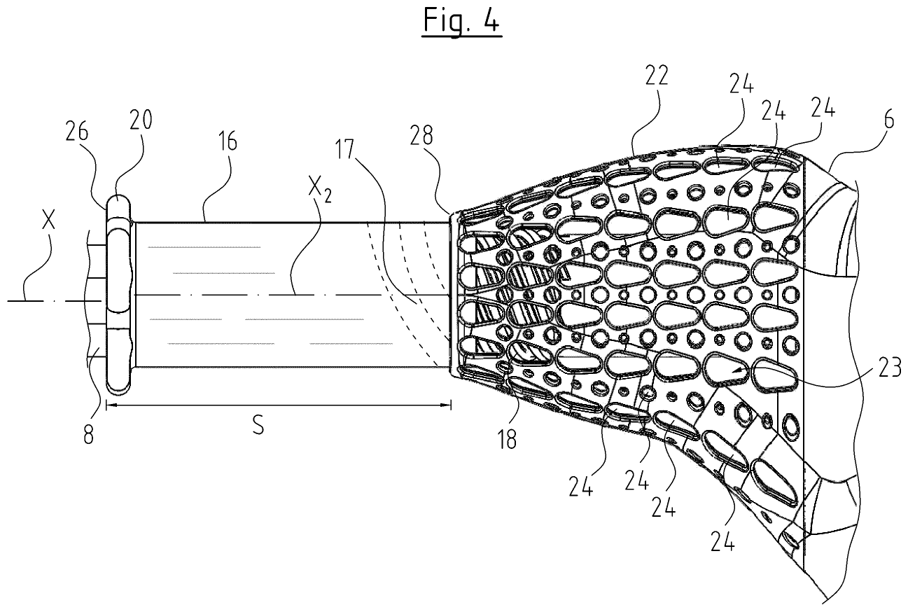

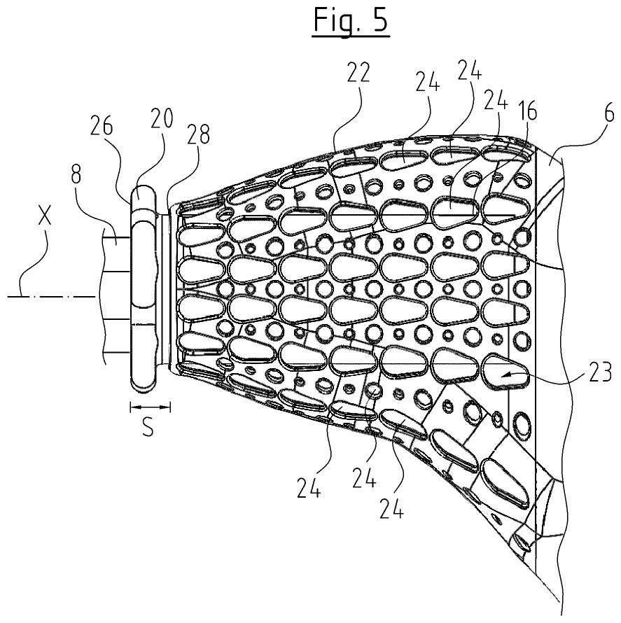

As is to be seen in FIG. 4, the section with the counter-thread 18 is situated in the handle 6 in the inside of a receiving space 23 which is surrounded or defined by a wall structure 22. The wall structure 22 comprises a plurality of openings 24, so that the wall structure 22 as a whole is perforated or is designed in the form of a grid-structure. The openings 24 permit the through-rising of the receiving space 23 and in particular the hygienic cleaning of the region of the counter-thread 18. The wall structure 22 is simultaneously designed in a rigid manner and has a shape, to the extent that it is possible to reliably grip the handle also in this region. The receiving space 23 which is formed in the inside by the wall structure 22 is open towards the distal end. The opening is an annular gap between the inner circumference of the wall structure 22 and the outer circumference of the endoscope shank 8. The spacer sleeve 16 extends out of the handle 6 in the distal direction parallel to the longitudinal axis X through this annular opening. The amount S by which the distal end 26 of the spacer sleeve 16 projects with respect to the distal end 28 of the handle 6 can be changed by the axial movement of the spacer sleeve 16 by way of rotating the thread 17 on the counter-thread 18. The spacer sleeve 16 is located at distal position in which the distance S is maximal in the views according to FIGS. 1, 3 and 4. In the position which is shown in FIG. 2 and FIG. 5, the spacer sleeve 16 is inserted completely into the inside of the handle, i.e. into the receiving space 23, so that the distance S is minimal.

The distal end of the spacer sleeve 16 forms a contact surface 26 which extends normally to the longitudinal axis X and the longitudinal axis X2. When the endoscopic instrument 4 is inserted into the working shank 2, this contact surface 26 comes to bear on a counter-contact surface which is formed by the proximal end of the working shank 2. The amount by which the endoscope shank 8 projects out of the distal end of the working shank 2 can therefore be changed via the change of the distance S. When the spacer sleeve 16 is extended completely out of the handle 6, i.e. the distance S is maximal, the distal end 30 of the endoscope shank 8 is located in a position in which it projects minimally with respect to the distal end 32, as is shown in FIG. 1. When the spacer sleeve 16 is moved completely into the handle 6, i.e. the distance S is minimal, then in contrast the distal end 30 of the endoscope shank 8 projects further with respect to the distal end 32 of the working shank 2, i.e. the endoscopic instrument 4 with the working shank 8 is positioned further distally in the working shank 2. Due to the fact that the grip region 20 is arranged at the distal end of the spacer sleeve 16, this region can be well gripped in every axial position and the axial positioning of the endoscope shank 8 in the working shank 2 can be easily changed on application of the endoscopic system by way of rotating the spacer sleeve 16.

While specific embodiments of the invention have been shown and described in detail to illustrate the application of the principles of the invention, it will be understood that the invention may be embodied otherwise without departing from such principles.

### LIST OF REFERENCE NUMERALS

2 working shank
4 endoscopic instrument
6 handle
8 endoscope shank
10 introduction opening
12, 14 rinsing/suction connections
16 spacer sleeve
17 thread
18 counter-thread
20 grip region
22 wall structure
23 receiving space
24 openings, perforation
26 distal end, contact surface
28 distal end of the handle
30 distal end of the endoscope shank
32 distal end of the working shank
X longitudinal axis
X₂ longitudinal axis, rotation axis
S distance

1. An endoscopic instrument comprising:
a handle;
an endoscope shank which extends distally from the handle; and
a spacer element which extends distally from the handle in the circumferential region of the endoscope shank and is linearly movably mounted in the handle.

2. An endoscopic instrument according to claim 1, wherein the spacer element is linearly movably mounted such that a distance of the spacer element to the distal end of the endoscope shank can be changed.

3. A endoscopic instrument according to claim 1, in combination with a working shank, wherein the spacer element comprises a contact surface which is configured to come into bearing contact with a proximal axial end of the working shank.

4. An endoscopic instrument according to claim 1, wherein the spacer element is configured as a spacer sleeve which circumferentially surrounds the endoscope shank.

5. An endoscopic instrument according to claim 1, wherein the spacer element comprises a thread which is in engagement with a counter-thread in the handle such that the spacer element is linearly movable by way of rotation of the thread.

6. An endoscopic instrument according to claim 5, wherein the thread is configured as an inner thread in the inside of the spacer sleeve.

7. An endoscopic instrument according to claim 5, wherein the counter-thread is configured as an outer thread on the outer circumference of the endoscope shank.

8. An endoscopic instrument according to claim 1, wherein the spacer sleeve comprises a grip region on a spacer sleeve outer periphery in a region of a spacer sleeve distal end.

9. An endoscopic instrument according to claim 1, wherein the handle comprises a wall structure which surrounds a receiving space which is open to the distal end of the handle and into which the spacer element engages.

10. A endoscopic instrument according claim 9, wherein the spacer element is movable linearly out of the receiving space and into the receiving space, in a manner such that the spacer sleeve projects differently far in the distal direction.

11. An endoscopic instrument according to claim 9, wherein the wall structure comprises through-openings and is configured as a grid structure.

12. An endoscopic system comprising:
an endoscopic instrument, the endoscopic instrument comprising a handle; an endoscope shank which extends distally from the handle; and a spacer element which extends distally from the handle in the circumferential region of the endoscope shank and is linearly movably mounted in the handle; and
a working shank into which the endoscopic instrument can be inserted from the distal end in a manner such that the spacer element comes to bear on the proximal end of the working shank.

13. An endoscopic instrument according to claim 12, wherein the spacer element is linearly movably mounted such that a distance of the spacer element to the distal end of the endoscope shank can be changed.

14. An endoscopic instrument according to claim 12, wherein the spacer element is configured as a spacer sleeve which circumferentially surrounds the endoscope shank.

15. An endoscopic instrument according to claim 12, wherein the spacer element comprises a thread which is in engagement with a counter-thread in the handle such that the spacer element is linearly movable by way of rotation of the thread.

16. An endoscopic instrument according to claim 15 wherein the counter-thread is configured as an outer thread on the outer circumference of the endoscope shank.

17. An endoscopic instrument according to claim 12, wherein the spacer sleeve comprises a grip region on a spacer sleeve outer periphery in a region of a spacer sleeve distal end.

18. An endoscopic instrument according to claim 12, wherein the handle comprises a wall structure which surrounds a receiving space which is open to the distal end of the handle and into which the spacer element engages.

19. A endoscopic instrument according claim 18, wherein the spacer element is movable linearly out of the receiving space and into the receiving space, in a manner such that the spacer sleeve projects differently far in the distal direction.

20. An endoscopic instrument according to claim 18, wherein the wall structure comprises through-openings and is configured as a grid structure.
